(19) United States
(12) Patent Application Publication
(10) Pub. No.: US 2016/0058373 A1
(43) Pub. Date: Mar. 3, 2016

(54) Running Energy Efficiency

(71) Applicant: Seiko Epson Corporation, Tokyo (JP)

(72) Inventor: Anand Kumar Anandabairavasamy, Richmond (CA)

(21) Appl. No.: 14/830,033

(22) Filed: Aug. 19, 2015

Related U.S. Application Data
(60) Provisional application No. 62/042,097, filed on Aug. 26, 2014.

(51) Int. Cl. A61B 5/00; A61B 5/11

(52) U.S. Cl. CPC A61B 5/4866; A61B 5/1122; A61B 5/7278; A61B 5/486; A61B 5/112; A61B 2503/10; A61B 2562/0219

(57) ABSTRACT

A motion analysis device uses an inertial sensor to calculate a measure of a user's athletic energy efficiency using only readings provided by the inertial sensor. The motion analysis device further provides suggestions to the user as to how to alter his posture or movements to improve his athletic energy efficiency. For example, in the case of a runner, the motion analysis device may instruct the runner to lean in one direction or another in order to alter his landing angle in such a way so as to improve his athletic energy efficiency. The device may further provide continuous feedback to the athlete to let him know when he is moving closer to, or away from, preset athletic energy efficiency goals.

### CROSS-REFERENCE TO RELATED APPLICATION(S)

U.S. Provisional Patent Application No. 62/042,097, filed Aug. 26, 2014, is hereby incorporated by reference in its/their entirety.

### BACKGROUND

1. Field of Invention

The present invention relates to the use of small scale inertial sensors to provide feedback information to an athlete. More specifically, the feedback information is geared toward identifying motion that may be adjusted to improve the athlete's energy consumption efficiency.

2. Description of Related Art

In recent years, analyzing the form and motion of sports has been gaining importance due to advancements in the development of small scale inertial sensors, such as an inertial measurement unit, i.e. IMU. An IMU contains a triad of gyroscopes and accelerometers, and can be attached to an athlete, or other subject, to aid in an analysis of the subject's motion. That is, information from the IMU can be used to analyze various postures and motion of the subject.

For example, in the category of track and field or marathon running, various parameters related to improving the performance of a typical runner can be obtained by analyzing data signals from one or more sensors, i.e. one or more IMU. Often, this information is analyzed in an effort to improve the runner's form, speed, and/or running technique.

While improvements in these areas are important, it would also be beneficial to identify areas of motion that an athlete can alter to improve his energy consumption efficiency. This would permit an athlete to improve his/her endurance, and thereby permit him/her to run further using the same amount available energy (i.e. without requiring additional rest or food).

### SUMMARY OF INVENTION

It is an object of the present invention to identify areas of motion that directly affect an athlete's energy use efficiency.

It is another object to identify parameters observable by small scale inertial sensors (e.g. inertial measurement units, IMU's) that can be related to parameters that affect an athlete's energy efficiency.

It is a further object of the present invention to identify energy related parameters of running, and to describe the use of small scale inertial sensors to derive and monitor those parameters.

The above objects are met with the use of an inertial measurement unit (IMU), which typically contain multiple gyroscopes and accelerometers that can be used to analyze various postures and motions of a subject. By attaching one or more small IMUs on an athlete, one may analyze the form and motion of the athlete and identify areas for improvement. For example, in the sports of track and field and marathon running, small IMU's can be used to provide information that can be analyzed to determine various parameters related to a runner's performance.

The present invention aims to improve a runner's efficiency. That is, it aims to help a runner achieve more work with less energy use. This is of particular importance to marathon runners, which need to conserve energy for long distance runs. Prior art attempts at providing a feedback system to improve efficiency merely provided an overall efficiency value to the runner, and it was hoped that the runner would try different strides and speeds in a blind effort to positively affect the efficiency reading. The present invention seeks to improve on this trial and error approach by identifying specific areas for improvement.

The present invention seeks to identify specific mechanisms of running that affect efficiency and provide information about these mechanisms, in addition to providing an overall efficiency ready. The present goal is to determine how altering these mechanisms affect the overall efficiency, and thereby provide specific suggestions for achieving greater overall efficiency.

Additionally an athlete may submit, to a device in accord with the present invention, hypothetical changes to theses mechanisms, and have the device determine how these hypothetical changes affect the energy efficiency. In this manner, an athlete may simulate several scenarios; each with different parameter changes until the athlete finds one that provides a desired efficiency. The athlete may then focus on adopting theses changes into his/her running technique.

A first step is to determine how to define a runner's efficiency. Athletic energy efficiency (AEE) may be defined as the amount of mechanical work (MW) done (or the metabolic workload) divided by the amount of metabolic energy (ME) expensed (or the energy used) to accomplish that work:

AEE=MW/ME (1)

The mechanical work MW, which is the product of an applied force over a given distance (e.g. displacement in a running direction), can be readily obtained from IMU readings. One way of measuring the metabolic energy ME expensed during a physical activity is to physically measure the athlete's oxygen intake during that activity. This approach, however, is cumbersome and limits its application. It is preferred that a measure of the expensed metabolic energy ME be instead obtained from the readings provided by an IMU, as is explained below.

Approximation formulas for oxygen consumption in ml/kg/min based on speed and time readings are provided in book Daniels' Running Formula by Jack Daniels. The needed speed and time reading are readily available using an IMU. The preferred embodiment of the present invention therefore uses these approximation formulas to determine oxygen intake, but it is to be understood that other formulation for determining oxygen consumption from IMU readings may also be used without deviating from the present invention. Irrespective of the formulas used to determined oxygen intake, the present invention uses the calculated oxygen intake value to calculate a measure of the expensed metabolic energy ME. In this manner, an overall athletic energy efficiency AEE can be obtained from IMU output readings, alone.

As is explained above, however, an aim of the present invention is to provide specifics regarding the mechanics of running that the athlete may focus on to improve efficiency. To do this, the invention turns to the first law of thermodynamics, which states that the change in the internal energy of a system (AU) is equal to the heat supplied to the system by its surroundings (Q) minus the work done by the system on its surroundings (W). This may be expressed as AU=Q−W.

A first step is thus to identify all the sources of change in energy in a runner, including both physiological and mechanical sources. The physiological source of energy change in the runner is the expensed metabolic energy ME. A source of mechanical energy change is in the spring potential energy (SP) of a runner as the runner takes a step.

Prior art sources provide motion models for simulating a foot-to-floor contact step, or running stride, that incorporate spring models. The present invention, however, does not focus on modeling a stride motion, but rather focuses on modeling the energy that is stored in the system and the amount of energy that is then released (i.e. energy change) as the runner takes a step. This may be equated to the potential energy stored in a spring prior to the spring being released. A spring coefficient (or spring constant) K is defined as K=(Applied Force-max)/(spring deformation (dz)). Force-max (in the z-direction, i.e. up-down or vertical direction) and dz (displacement in the z-direction, i.e. up-down or vertical direction) can be obtained from IMU readings. It is to be understood that the Force-max and dz may be observed during landing in a running stride or when the runner's body is in contact with the ground (i.e. when a foot is on the ground) during a running stride/step. The spring potential energy SP can then be determined as SP=½K(dz)². The IMU can thus be used to determine the spring potential energy SP.

The first law of thermal dynamics may be restated using the present energy, heat and work parameters as follows (allowing for change in sign due to the direction of energy/work/heat flow into and out of the system):

expensed Metabolic Energy (ME)+Spring Potential Energy (SP)=Mechanical Work done (MW)+Heat Dissipation (HD)

The expensed metabolic energy ME can thus be defined as:

ME=MW+HD−SP (2)

Referring to equation (1) above, it can be seen that the athletic energy efficiency AEE can be increased by decreasing the expensed metabolic energy ME. As is evident from equation (2), ME may be decreased by increasing the spring potential energy SP and/or by decreasing the heat dissipation HD, which is wasted energy. The problem now becomes how to reduce heat dissipation while running.

The present invention identifies mechanical sources of heat dissipation HD. Physiological sources may already be accounted for by the expensed metabolic energy ME. Some of the mechanical sources (i.e. parameters) that have a relationship to heat dissipation HD are Contact Time (T_{c}), Landing Angle (L_{A}) and the Spring Coefficient (K). Contact Time (T_{c}) is the time a particular foot is in contact with the ground. The landing angle L_{A} is the angle of the center of mass to the ground at the time of landing. Contact time T_{c} may be measured by analyzing the accelerometer values and their zero crossing points. Landing angle L_{A} may be obtained by measuring the horizontal and vertical displacements of the body during the contact time T_{c}. By modeling these sources using correlation coefficients, an estimate of the heat dissipation HD can be obtained as a function of these parameters, as follows:

HD=a₁T_{c}+a₂L_{A}+a₃K (3)

Values a₁, a₂, a₃ are the correlation coefficients, and parameters T_{c}, L_{A}, and K can be monitored in real time using the inertial measurement unit (IMU), and the runner can be advised to change these parameters to reduce heat dissipation, and thereby improve energy efficiency.

The heat dissipation HD can also be estimated from ME, SP and MW using formula (2) as follows:

HD=ME+SP−MW (4)

Alternatively as is stated above, HD can be estimated from direct measurements of the volume of oxygen intake. Correlation coefficients a1, a2, and a3 can be estimated through regression analysis. For example, if a person's oxygen intake is physically measured directly such that HD is determined directly, then coefficients a1, a2, and a3 can be determined from the measured parameters T_{c}, L_{A}, and K and the observed value of HD. Alternatively, HD may be obtained indirectly from formula (4). Therefore, coefficients a1, a2, and a3 may be personally calibrated to a specific individual. Alternatively, coefficients a1, a2, and a3 may be assigned values extracted from known typical ranges.

A runner can also submit hypothetical changes to any of the above parameters and run a simulation to identify his/her ideal parameter conditions for improved efficiency. The IMU can then signal the runner to let him/her know whether he/she is getting closer to his/her set goals. These signals may be in the form of a visual display, audible signal, or a physiological indicator such as causing the IMU to vibrate faster as the runner moves away from the set goals and slowing down and eliminating vibration as the runner moves closer to set goals. Alternatively, changes in vibration may indicate whether the runner is moving from set goals in one direction or another. For example, should the runner lean his/her landing angle toward the left or toward the right to improve his/her efficiency.

The present objects are also met in a motion analysis device including: a data processing unit that uses output from an inertial sensor to obtain a metabolic workload of a test subject while running and a measure of energy used by the test subject while running; and a calculation processing unit that uses the metabolic workload and the measure of energy used to obtain an athletic energy efficiency of the test subject.

Preferably, the output from the inertial sensor includes acceleration data and distance moved while running of the test subject; and the metabolic workload is obtained by a product of the distance moved while running and a first force measure, the first force measure being obtained from the weight of the test subject and the acceleration data in the running direction.

Further preferably, the measure of energy used is determined based on the running speed calculated from the output of the inertial sensor and the running time. In this case, the running speed and the running time may be used to calculate oxygen intake, and the energy used may be obtained from the calculated oxygen intake.

Additionally, the energy used may be obtained by subtracting a calculated spring potential energy of the test subject from a sum of the metabolic workload and a measure of heat dissipation caused by running; the spring potential energy of the test subject may be calculated from a spring constant of the subject that is determined by obtaining a maximum vertical force defined as a product of the weight of the test subject and a maximum vertical acceleration of the test subject as determined from the inertial sensor, and dividing this maximum vertical force by a maximum vertical displacement of the test subject while the test subject touches the ground during a running stride, this maximum vertical displacement also being provided by the inertial sensor.

Additionally, the measure of heat dissipation may be obtained based on a landing time and a landing angle of the test subject's leg, and the spring constant.

The motion analysis device may further include a report processing unit 31 that generates a report signal communicating the athletic energy efficiency to the test subject. Preferably, the report processing unit 31 compares the obtained athletic energy efficiency with a pre-specified target value, and generates the report signal based on a result of the comparison.

The present objects are also met in a motion analysis method including: a step of obtaining a metabolic workload of a test subject while running, the metabolic workload being obtained using an output from an inertial sensor; a step to obtaining a measure energy used by the test subject while running; and a step to obtaining an athletic energy efficiency of the test subject by using the metabolic workload and the measure of energy used.

The present objects are also met in a motion analysis system including: an inertial sensor attachable to a test subject and configured to acquire inertial data of the test subject while running; a data processing unit that uses outputs from the inertial sensor to obtain a measure of metabolic workload of the test subject while running and a measure of energy used by the test subject while running; a processing unit that uses the measure of metabolic workload and the measure of energy used to obtain an athletic energy efficiency of the test subject.

Preferably, the motion analysis system further includes a reporting unit that communicates the obtained athletic energy efficiency to the test subject.

Other objects and attainments together with a fuller understanding of the invention will become apparent and appreciated by referring to the following description and claims taken in conjunction with the accompanying drawings.

### BRIEF DESCRIPTION OF THE DRAWINGS

In the drawings wherein like reference symbols refer to like parts.

FIG. 1 defines a runner's gait cycle.

FIG. 2 illustrates a spring model for determining a spring coefficient.

FIG. 3 illustrates positive work action, which may be denoted as MW+.

FIG. 4 illustrates negative work action, which may be denoted as MW−.

FIG. 5 illustrates a method of calculating the landing angle L_{A}, i.e. θ.

FIG. 6 shows a model/formula for estimating a volume of oxygen consumption.

FIG. 7 illustrates a model/formula for identifying a point of maximum (i.e. saturated) oxygen consumption VO₂ₘₐₓ while running.

FIG. 8 illustrates equations for calculating energy used EU (i.e. energy consumption) from the amount of oxygen used (e.g. VO or VO₂ₘₐₓ) determined using the equations of FIG. 6 or 7.

FIG. 9 illustrates a definition for energy efficiency.

FIG. 10 illustrates an expression for energy efficiency.

FIG. 11 provides an expression for calculating a heat dissipation value HD using parameters determinable using an IMU.

FIG. 12 provides an expression for estimating heat dissipation HD.

FIG. 13 illustrates some of the equations that can be determined from IMU data readings to obtain needed parameters.

FIG. 14 provides an overview of the present invention.

FIG. 15 provides an alternate embodiment of the present invention.

### DESCRIPTION OF THE PREFERRED EMBODIMENTS

In recent years, analyzing the form and motion of sports has been gaining importance due to advancements in the development of small scale inertial sensors. An example of a small scale inertial sensor is an inertial measurement unit, i.e. IMU, which typically contains a triad of gyroscopes and accelerometers. An IMU can be used to analyze the posture and motion of a subject. In the sport categories of track and field and marathon running, various parameters that relate to improving the performance of a typical runner can be obtained by analyzing signals from such sensors. As an exemplary implementation of the present invention, a preferred embodiment of the present invention discusses energy related aspects, or parameters, of running and the use of sensors in deriving those parameters.

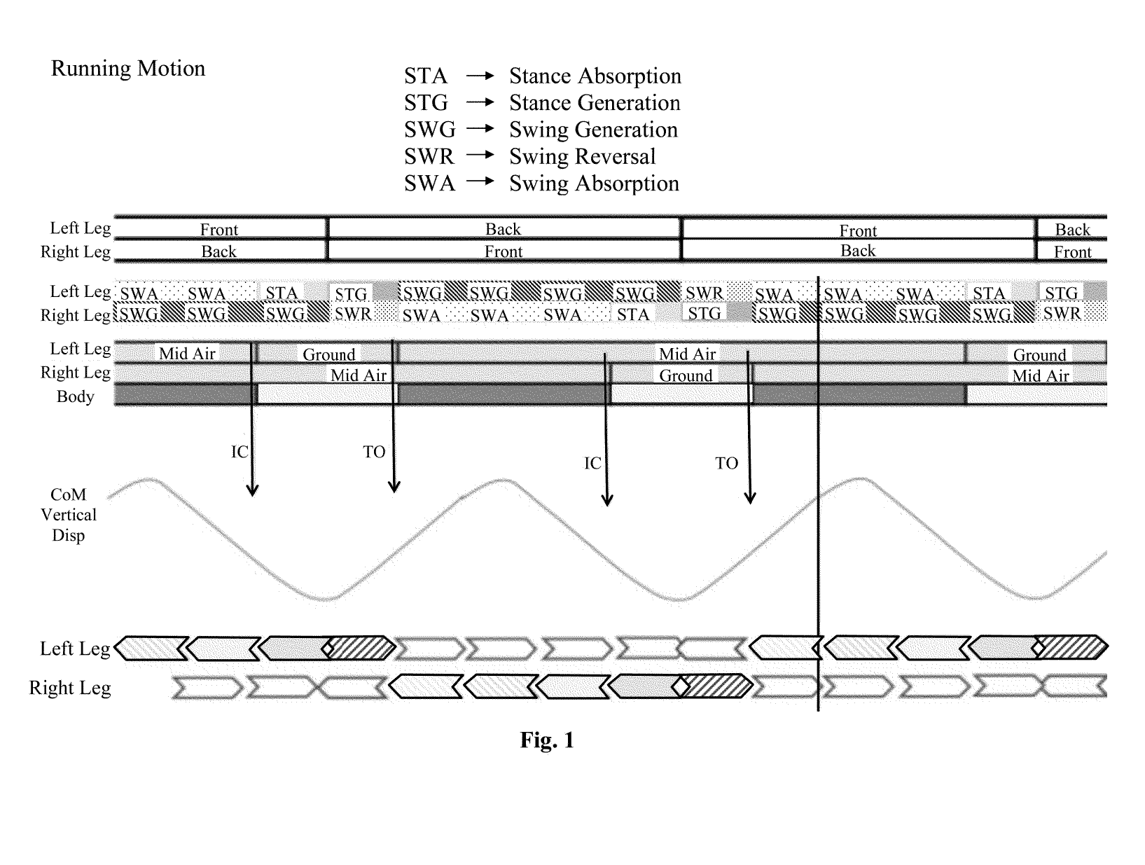

A running activity can be typically broken down into a sequence of gaits, where each gait is defined as a pair of left and right strides. From existing literature, the gait cycle can be defined as shown in FIG. 1.

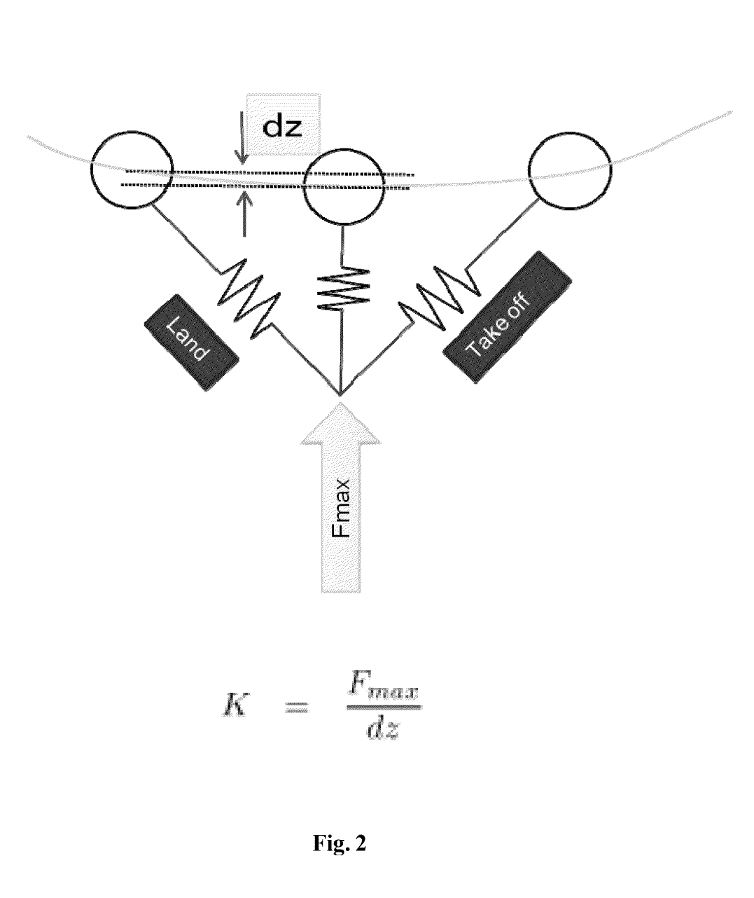

The ground contact portion of the gait may be approximated as a spring mass model, as is illustrated in FIG. 2. The muscles and tendons in the lower limbs act like a spring, storing energy during landing and releasing this energy during toe off from the ground. A vertical spring coefficients (K) can then be calculated from the force (e.g. Fmax) and the vertical displacement (e.g. dz). Traditionally, force plates on the ground were used to collect the force data. This had its limitations. For example, the data could only be collected for a few instances. With the use of small size IMU's, the force impacted on the body can be sensed continuously. This provides a greater advantage in monitoring key parameters of running and improving upon them. In this manner, the spring coefficient may be defined as K=(Fmax)/(dz).

The spring potential energy SP, i.e. the energy stored and released by the spring, may be defined as (considering an ideal spring): SP=½K(dz)².

The IMU can also be used to determine mechanical work, MW. That is, since the IMU can be used to derive the position (x), velocity (v) and acceleration (a) vectors, the mechanical work MW done while running can be calculated using the following formula:

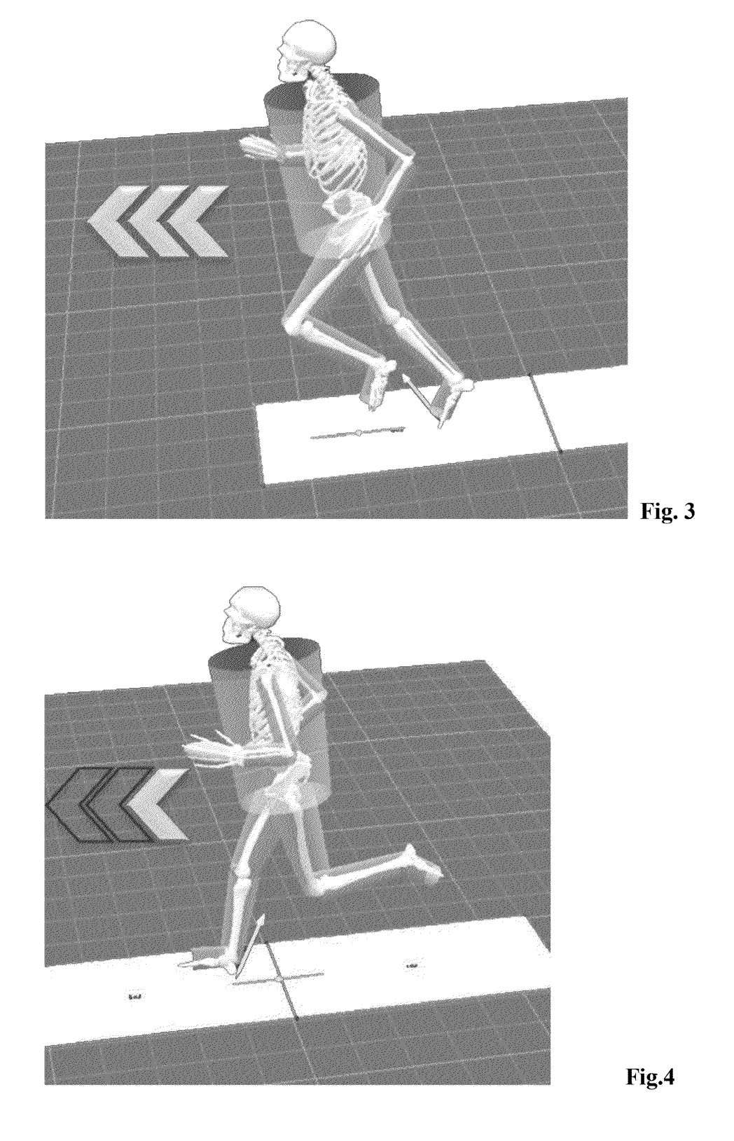

MW=∫(F·v)dt

where F=ma (i.e. force vector equals mass (or colloquially a test subject's weight on the surface of the earth) times acceleration vector) is the force vector acting on the body and v is the velocity vector of the body. Positive work may be denoted by MW+ and negative work by MW−. An example of positive work while running is illustrated in FIG. 3, and an example of negative work while running is illustrated in FIG. 4.

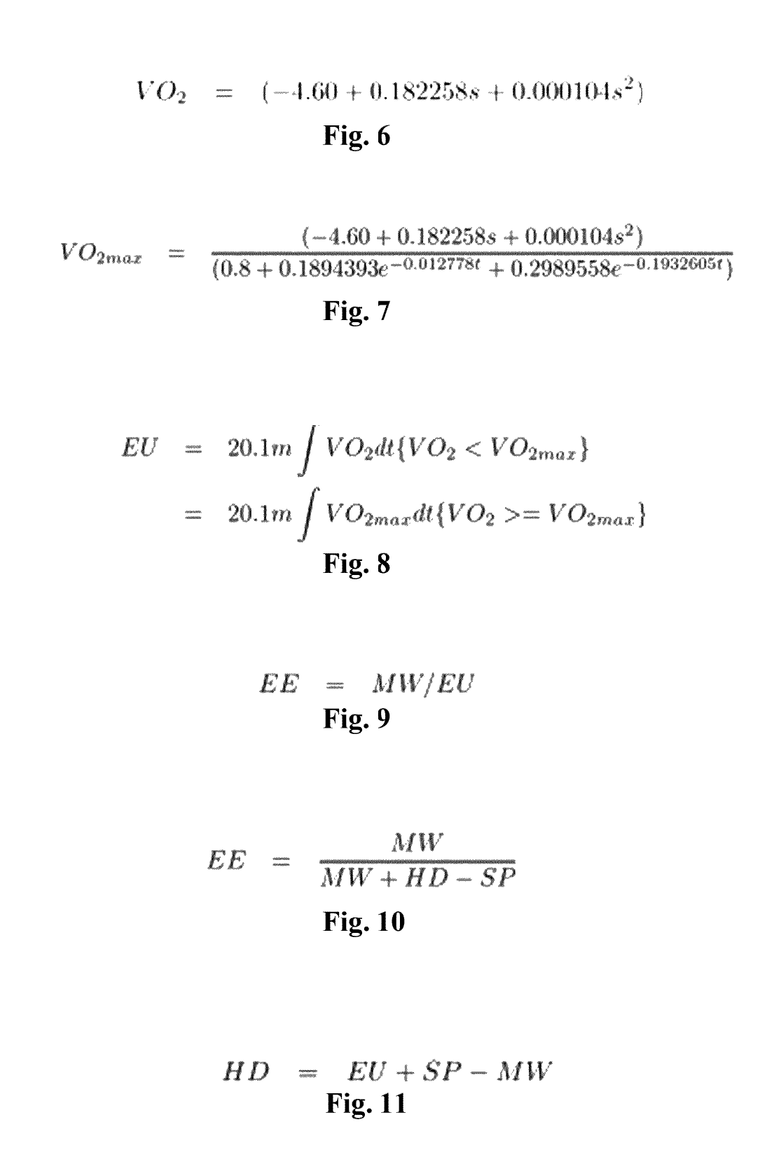

Energy consumption may be monitored indirectly. In the past, oxygen intake has been used as a base to model energy consumption during running. As it is currently impractical to directly measure the amount of oxygen intake while running a marathon on a standard running track, an approximation that uses parameters monitored by an IMU is a more practical solution. In his book, Daniels' Running Formula, author Jack Daniels suggests a model for estimating a volume of oxygen consumption (VO) while running. Each ml of 02 is approximated to 20.1 Joules of energy. This basic model is illustrated in FIG. 6.

Also, the oxygen consumption will saturate at a particular value (i.e. a maximum oxygen volume point, VO₂ₘₐₓ) beyond which there will be no increase in the amount of oxygen consumed. This maximum value VO₂ₘₐₓ is defined by the formula illustrated in FIG. 7. In this formula, “s” denotes the speed in m/min and t is the time in min. The unit of oxygen consumption is ml/kg/min.

With a measure of oxygen consumption thus determined, an estimation of energy consumption (e.g. the energy used, EU) can be obtained. That is, the energy used EU is calculated from the above VO values of FIGS. 6 and 7. VO₂ₘₐₓ is calculated based on the best performance of the runner, and this value is stored. To calculate the actual energy used, the VO₂ value is first calculated and compared to the VO₂ₘₐₓ value. If the calculated VO₂ value exceeds the saturation point VO₂ₘₐₓ, then VO₂ₘₐₓ is used to calculate EU; otherwise VO₂ is used to calculate EU, as is illustrated in the conditional equations of FIG. 8.

With energy consumption EU thus determined, one may then turn to the question of energy efficiency. An efficient runner would maximize the work done while consuming minimal energy. Thus, energy efficiency EE is herein defined as the ratio of mechanical work MW done to the energy used EU, as is illustrated in FIG. 9.

The following expression can be defined from thermal dynamics:

Metabolic energy produced (EU)+Spring Potential Energy (SP)=Mechanical Work Done (MW)+Heat Dissipation (HD)

Based on the above expression, energy use EU (i.e. metabolic energy produced) can be defined as EU=MW+HD−SP. Therefore, the energy efficiency EE of FIG. 9 may be rewritten as illustrated in FIG. 10. From FIG. 10, it can be seen that to obtain higher efficiency, a runner should increase the spring potential while at the same time decreasing the heat energy (e.g. heat dissipation HD), which is a wasted energy.

From the IMU signals, one can calculate the MW, SP and EU values. Thus, combining the equations of FIGS. 9 and 10, heat dissipation value HD can be calculated as illustrated in FIG. 11.

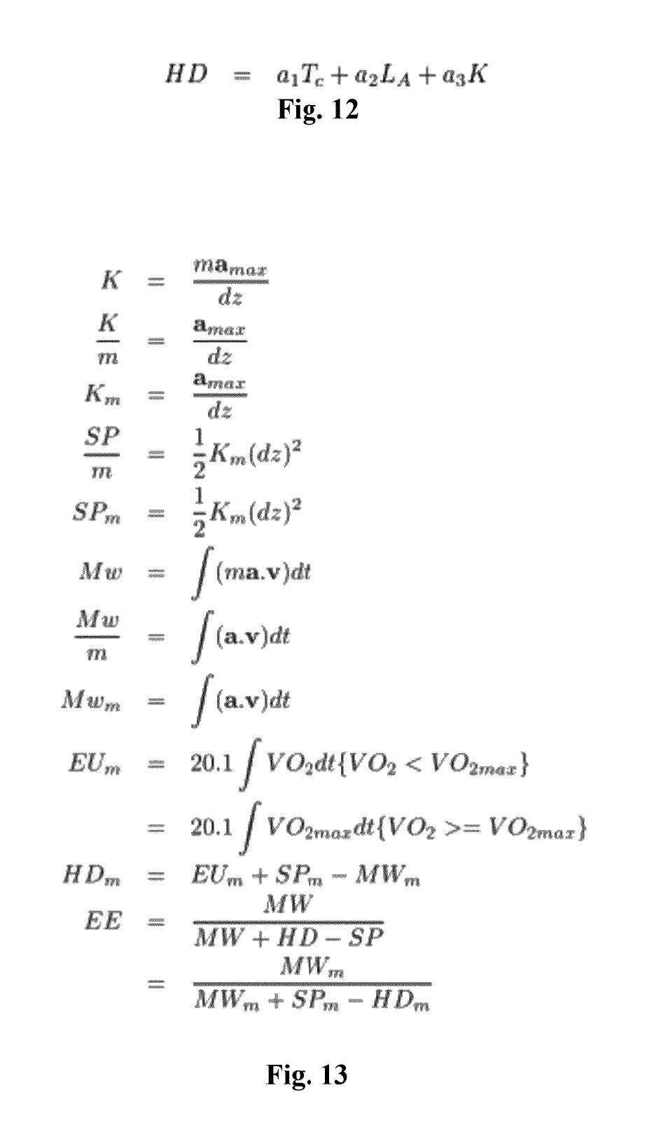

For a runner to be efficient, the runner needs to reduce the amount of heat energy dissipation, which is a wasted energy. To achieve this, one needs to identify running-related parameters that have a relationship to heat energy dissipation. Some running-related parameters found to have a relationship to heat dissipation are Contact Time (Tc), Landing Angle (L_{A}) and Spring Coefficient (K). By modeling this relationship using correlation coefficients, the heat dissipation HD can be estimated as a function of these variables, as is illustrated in FIG. 12.

In FIG. 12, a₁, a₂, a₃ are correlation coefficients. T_{c} is the Contact Time, L_{A} is the Landing Angle and K is the spring constant. The T_{c}, L_{A}, K values can be monitored in real-time using IMU signals, and the runner can be advised to change these parameters so that there is minimal heat dissipation, thereby improving energy efficiency.

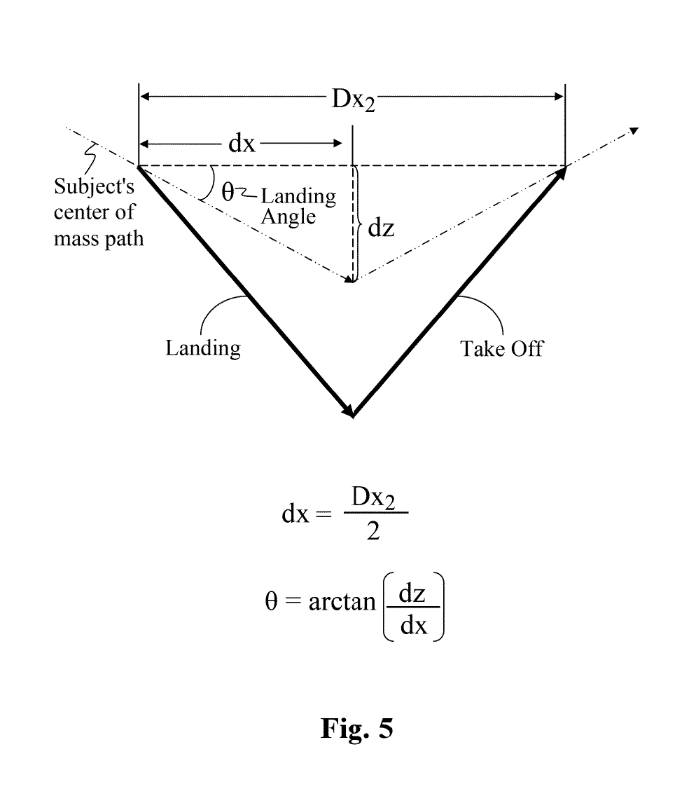

With reference to FIG. 5, contact time T_{c} is the time a particular foot is in contact with the ground, which may be determined by observing the center of mass path, as determined from the IMU. For example, contact time T_{c} may be measured by analyzing the accelerometer values and their zero crossing points. The landing angle L_{A} may be obtained by measuring the horizontal displacement (Dx₂) and vertical displacements (dz) of the body during the contact time. For illustration purposes, the landing angle is identified as θ in FIG. 5, and it can be described as the angle of the center of mass of the subject (i.e. the body) to the ground at the time of landing. This angle θ can be determined as the arctan of (dz/dx), where dx is half the forward motion of the center of mass (i.e. horizontal displacement (Dx₂)) during the time that the foot is in contact with the ground. Vertical displacement dz is a measure of the body compression during landing, as is explained above in the discussion of the spring model of a runner's gate.

Alternatively if one wishes to avoid the use of the spring potential K, energy efficiency EE and heat dissipation HD can also be calculated using only positive work values as follows:

HD=(EU)−(MW+)

and

EE=(MW+)/[(MW+)+(HD)]

Where MW+ denotes the positive work component as defined above.

The above calculations need the force vector to be calculated which in turn needs the mass (or weight on the surface of the earth) of the runner to be input. The mass information of the runner may be avoided by using equations based on the unit mass, i.e. unit mass equations, which provide answers on a per mass basis and thus do not require that the runner's mass be input.

Some of the equations that can be determined from IMU data readings to obtain needed parameters, including unit mass equations, are illustrated in FIG. 13.

A runner can also submit hypothetical changes to any of the above parameters and run a simulation to identify his/her ideal parameter conditions for improved efficiency. The IMU can then signal the runner to let him/her know whether he/she is getting closer to his/her set goals. These signals may be in the form of a visual display, audible signal, or a physiological indicator such as causing the IMU to vibrate faster as the runner moves away from the set goals and slowing down and eliminating vibration as the runner moves closer to set goals. Alternatively, changes in vibration may indicate whether the runner is moving from set goals in one direction or another. For example, should the runner lean his/her landing angle toward the left or toward the right.

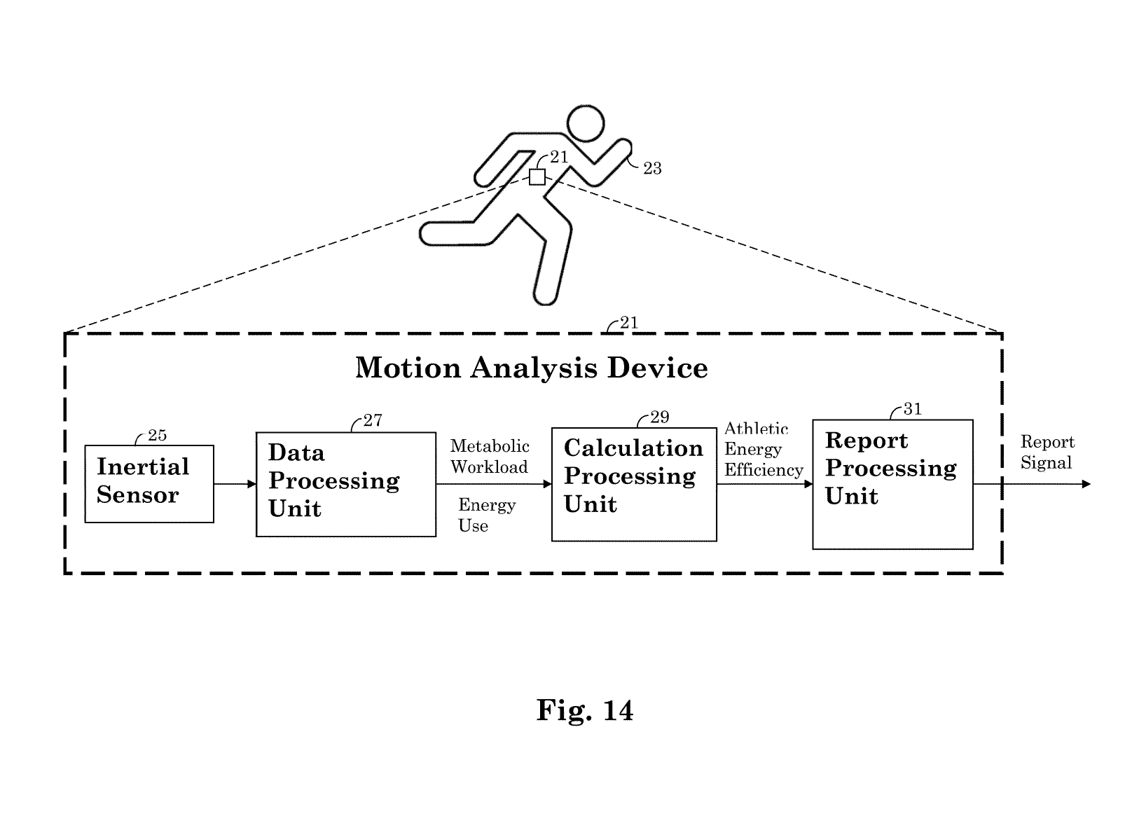

FIG. 14 provides an overview of the present invention in use. The present example shows a motion analysis device 21 wearable on a test subject (i.e. runner or user) 23. In the present example, motion analysis device 21 is preferably positioned close to the center of mass (or center of movement to be analyzed) of the user's body. Since in the present example, the motion analysis device 21 is used while running and the movement to be analyzed is the overall movement of the user's entire body as he runs, motion analysis device 21 is shown preferably positioned on the user's torso.

Motion analysis device 21 includes an inertial sensor, as described above, and further preferably includes a data processing unit 27 to perform many of the above-described computations. It is to be understood that data processing unit 27 is an electronic computing device (such as a CPU, ASIC, FPLD, PLA, PLD, etc.), and it preferably receives inertial readings from inertial sensor 25 and uses these inertial readings to obtain a metabolic workload of the test subject (i.e. user 23) while the test subject 23 is running and obtain a measure of energy used by the test subject while the test subject 23 is running.

A calculation processing unit 29 uses the metabolic workload and the measure of energy used to obtain an athletic energy efficiency of the test subject 23. A report processing unit 31 may then be used to generate a report signal communicating the athletic energy efficiency obtained by calculation processing unit 29 to the test subject 23. This report signal may be in the form of an audible tone, a physical movement such as vibration speed, or a text file, or other form of file (i.e. image information).

It is to be understood that calculation processing unit 29 and data processing unit 27 may be integrated into a single electronic device or chip (CPU, ASIC, FPLD, PLA, PLD, etc.). That is, calculation processing unit 29 may be incorporated into data processing unit 27. Similarly, the report processing unit 31 and calculation processing unit 29 may be incorporated into data processing unit 27.

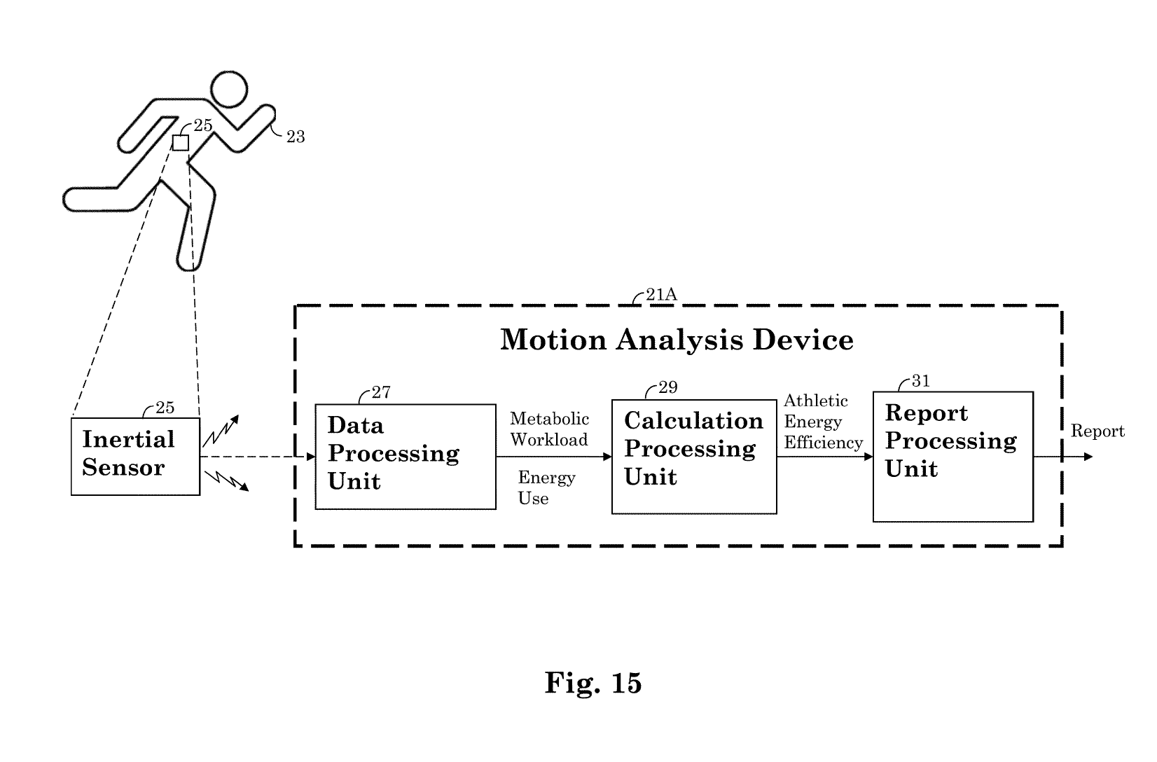

FIG. 15 provides an alternate embodiment of the present invention wherein all elements similar to those of FIG. 14 have similar reference characters and are described above. In the embodiment of FIG. 15, the inertial sensor 25 is maintained separate from motion analysis device 21A. In this manner, inertial readings from inertial sensor 25 may be transmitted (via a wired connection or wirelessly) to motion analysis device 21A. For example, inertial sensor 25 may be positioned as described above in reference to FIG. 14 to monitor a specific motion of interest (e.g. on the torso to monitor a running motion), and its readings sent via a wireless communication link, such as the bluetooth technology standard known in the art, to motion analysis device 21A. In this case, motion analysis device 21A may be embodied by a program (or application) running on a remote, general computing device or on specialized equipment. In a preferred embodiment, motion analysis device 21A may be embodiment by smart phone running a so-called “app” (i.e. software application) having instructions to execute a method in accord with the present invention to achieve the above-described function.

While the invention has been described in conjunction with several specific embodiments, it is evident to those skilled in the art that many further alternatives, modifications and variations will be apparent in light of the foregoing description. Thus, the invention described herein is intended to embrace all such alternatives, modifications, applications and variations as may fall within the spirit and scope of the appended claims.

What is claimed is:

1. A motion analysis device comprising:
a data processing unit that uses output from an inertial sensor to obtain a metabolic workload of a test subject while running and a measure of energy used by the test subject while running; and
a calculation processing unit that uses the metabolic workload and the measure of energy used to obtain an athletic energy efficiency of the test subject.

2. The motion analysis device of claim 1, wherein:
the output from the inertial sensor includes acceleration data and distance moved while running of the test subject; and
the metabolic workload is obtained by a product of the distance moved while running and a first force measure, the first force measure being obtained from the weight of the test subject and the acceleration data in the running direction.

3. The motion analysis device of claim 1, wherein the measure of energy used is determined based on the running speed calculated from the output of the inertial sensor and the running time.

4. The motion analysis device as described in claim 3, wherein the running speed and the running time are used to calculate oxygen intake, and the energy used is obtained from the calculated oxygen intake.

5. The motion analysis device of claim 2, wherein:
the energy used is obtained by subtracting a calculated spring potential energy of the test subject from a sum of the metabolic workload and a measure of heat dissipation caused by running;
the spring potential energy of the test subject is calculated from a spring constant of the subject that is determined by obtaining a maximum vertical force defined as a product of the weight of the test subject and a maximum vertical acceleration of the test subject as determined from the inertial sensor, and dividing this maximum vertical force by a maximum vertical displacement of the test subject while the test subject touches the ground during a running stride, this maximum vertical displacement also being provided by the inertial sensor.

6. The motion analysis device of claim 5, wherein the measure of heat dissipation is obtained based on a landing time and a landing angle of the test subject's leg, and the spring constant.

7. The motion analysis device of claim 1, further comprising a report processing unit that generates a report signal communicating the athletic energy efficiency to the test subject.

8. The motion analysis device of claim 7, wherein the report processing unit compares the obtained athletic energy efficiency with a pre-specified target value, and generates the report signal based on a result of the comparison.

9. A motion analysis method comprising:
a step of obtaining a metabolic workload of a test subject while running, the metabolic workload being obtained using an output from an inertial sensor;
a step to obtaining an measure of energy used by the test subject while running; and
a step to obtaining an athletic energy efficiency of the test subject by using the metabolic workload and the measure of energy used.

10. A motion analysis system comprising:
an inertial sensor attachable to a test subject and configured to acquire inertial data of the test subject while running;
a data processing unit that uses outputs from the inertial sensor to obtain a measure of metabolic workload of the test subject while running and a measure of energy used by the test subject while running;
a processing unit that uses the measure of metabolic workload and the measure of energy used to obtain an athletic energy efficiency of the test subject.

11. The motion analysis system of claim 10, further comprising a reporting unit that communicates the obtained athletic energy efficiency to the test subject.
